(19) United States
(12) Patent Application Publication
(10) Pub. No.: US 2022/0395497 A1
(43) Pub. Date: Dec. 15, 2022

(54) Cancer Treatment Using Camptothecin Derivatives

(71) Applicant: Vivacitas Oncology, Inc., Walnut Creek, CA (US)

(72) Inventor: Elise BROWNELL, Lafayette, CA (US)

(73) Assignee: Vivacitas Oncology, Inc., Walnut Creek, CA (US)

(21) Appl. No.: 17/776,657

(22) PCT Filed: Nov. 19, 2020
(86) PCT No.: PCT/US2020/061199

§ 371 Date: May 13, 2022

Related U.S. Application Data
(60) Provisional application No. 62/937,981, filed on Nov. 20, 2019.

(51) Int. Cl. A61K 31/4745; A61P 35/00; A61K 47/44; A61K 9/00

(52) U.S. Cl. CPC A61K 31/4745; A61P 35/00; A61K 47/44; A61K 9/0019

(57) ABSTRACT

A method of treating cancer in a patient by administering to a patient a composition comprising a therapeutically effective amount of AR-67, wherein the method is at least as effective as the administration of a dose of other camptothecins delivered alone or as a combination therapy, and wherein the method reduces the incidence of one or more adverse events.

### CROSS-REFERENCE TO RELATED APPLICATIONS

This application claims priority to and the benefit of U.S. Provisional Patent Application No. 62/937,981 filed Nov. 20, 2019, which is hereby incorporated by reference in its entirety.

### FIELD OF THE INVENTION

The present invention relates to methods for treating cancer.

### BACKGROUND OF THE INVENTION

AR-67 [(205)-7-tert-butyldimethysilyl-10-hydroxycamptothecin] is a third generation camptothecin analog currently under development by Vivacitas Oncology. AR-67 was first discovered and described in U.S. Pat. No. 6,136,978, and a new method of synthesis is described in U.S. Pat. No. 9,447,126, the disclosures of which are incorporated by reference. The structure of AR-67 is shown in FIG. 1. AR-67 belongs to a class of anticancer drugs that inhibit DNA topoisomerase I (TopoI), a ubiquitous and essential mammalian nuclear enzyme that relaxes DNA supercoiling generated by transcription, replication and chromatin remodeling. See Rothenberg, M L. 1997, Annal. Oncology, 8:837-855; Abang, A M. 1998, Semin Hematol. 35:13-21; Pommier, Y. 2006, Nat Rev Cancer, 6(10):789-802. Camptothecin is a naturally occurring product, originally found in the Chinese tree Camptotheca acuminate. The National Cancer Institute (NCI) first discovered the antitumor activity of camptothecin in the 1960's. In 1985, TopoI was shown to be the molecular target of camptothecin. See, Hsiang, Y K, et al. 1985, J. Biol. Chem. 260: 14873-14878.

A significant step for camptothecin interaction with DNA is stabilization of the enzyme TopoI/DNA complex and cleaving of the DNA to allow for uncoiling. In the presence of camptothecin, a stable enzyme complex is formed which is reversible and nonlethal. However, the single-strand DNA breaks become irreversible double-strand breaks when the DNA replication fork collides with the reversible enzyme complex during S phase or during unscheduled DNA replication. Therefore, the cytotoxic effect of the camptothecins requires active DNA replication. In vitro studies have demonstrated that cells in the S phase of the cell cycle are 100 to 1,000 times more sensitive to camptothecin-induced apoptosis than cells in the G1 or G2 phase of the cell cycle. See Li, RH, et al. 1972, Cancer Res. 32:2643-2650.

As a class of drugs, the camptothecins exhibit unique dynamics and reactivity in vivo, both with respect to drug hydrolysis and blood protein interactions. These factors have confounded their pharmaceutical development and clinical implementation. In terms of hydrolysis, each of the clinically relevant camptothecins contains an α-hydroxy-δ-lactone pharmacophore. At physiologic pH this functionality is highly reactive and readily hydrolyzes to the “ring opened” carboxylate form.

Thus, as a result of this reversible ring-opening, camptothecins exist in an equilibrium of two distinct drug species: 1) the biologically active form where the lactone ring remains closed; and 2) the biologically-inactive carboxylate form generated by the hydrolysis of the lactone ring of the parent drug. Serum protein interactions for specific camptothecin analogs (camptothecin and 9-aminocamptothecin) further compound this problem, as the carboxylate form of the drug binds human serum albumin with high affinity and thereby drives the lactone/carboxylate equilibrium to favor the formation of the carboxylate form. See Burke, T G, et al. 1993, Anal. Biochem. 212: 285-287; Burke, T G et al. 1993, J Med. Chem. 36:2580-2582. Historically the carboxylate form of the camptothecin agent has been considered inactive. See Jaxel, C. et al. 1993, Cancer Res. 49: 1465-1469. However, structural studies suggest that both forms bind to DNA. See Staker, B L, et al. 2002, Proc. Nat. Acad. Sci. USA 99:15387-15392. A stable lipophilic form that would diffuse into the cells through the lipid bilayer has been considered a desirable molecular trait.

There has been a significant effort to develop novel camptothecin analogs with improved stability and activity profiles. These efforts resulted in the FDA approval of two camptothecin analogs, topotecan (Hycamtin GlaxoSmithKline) and the prodrug irinotecan (Camptosar Pfizer). Irinotecan is metabolized by carboxylesterases to form the active agent SN-38. Currently, topotecan is approved as a second line therapy for ovarian and small cell lung cancer, while irinotecan (CPT-11, active metabolite), in conjunction with 5-fluorouracil (5-FU), is approved either as a treatment for colon cancer or alone as a salvage therapy for 5-FU refractory colon cancer. The ratios of the lactone AUC to total AUC for topotecan and SN-38 are 0.37 and 0.51, respectively. See Wall, J G, et al. 1992, Anticancer Drugs 3:337-345; van Warmerdam, L G et al. 1996 J. Clin. Oncology 11: 2194-2204; Rothenberg, M L, et al. 1993, Cancer Research 59:L4898-4905. Both products display a limited spectrum of clinical activity, therefore additional research effort has been directed toward the development of third generation analogs.

Table 1 outlines the camptothecin derivates in current clinical use. See Pollack, IF, et al. 2007 Cancer Res. 59: 4898-4905

TABLE 1
Camptothecins in Clinical Use

| Camptothecin derivative | Status | Remarks | Indications |
|---|---|---|---|
| Topotecan | FDA approved | Intravenous infusion | Metastatic ovarian cancer |
| hydrochloride | (GlaxoSmithKline) | (water-soluble) | (second line); SCLC (second |
| (Hycamptin) | | | line) |
| Irinotecan | FDA approved | Intravenous infusion | Metastatic colorectal cancer |
| hydrochloride | (Pfizer*) | (water-soluble) | (first line with 5FU/ |
| (Camptosar) | | | leucovorin) |
| Lurtotecan GI- | Phase II | Intravenous | Ovarian and other carcinomas |
| 147211 NX211 | (Gilead [symbol] ) | infusion; liposomal | |
| | | (NX 211) | |
| Gimatecan (ST-1481) | Phase I/II | Oral administration; | Glioblastoma, SCLC and solid |
| | (Novartis^{§}) | lipophilic | tumors |
| PEG- | Phase II | PEGylated | NSCLC and other solid |
| camptothecin; | (Enzon Inc.) | derivative; | tumors |
| Prothecan | | intravenous infusion | |
| | | (water-soluble) | |
| Karenitecin; BNP-1350 | Phase II | Oral administration; | Glioblastoma, melanomas and |
| | (Bionumerik | lipophilic | NSCLC and ovarian cancer |
| | Pharmaceuticals) | | |
| AR-67 | Phase II | Lipophilic | Glioblastoma and |
| | (Arno) | | Myelodysplastic Syndrome |
| Diflomotecan; BN 80915 | Phase II | Intravenous infusion | Advanced metastatic cancers; |
| | (Ipsen) | | colon, breast, prostate and |
| | | | small cell lung cancer |

*Licensed from Yakult Honsha Co. Ltd, Japan, and Daiichi Pharmaceutical Co. Ltd, Japan. [symbol]
Licensed from Glaxo Wellcome.
^{§}Licensed from Sigma-Tau.
¶Carboxy methyldextran polyalcohol carrier covalently linked through a peptidyl spacer.

In 1996, a joint collaborative research effort was undertaken between the University of Kentucky and the University of Pittsburgh with the goal of designing camptothecins with improved human blood stability. Lactone stabilization was achieved by introducing substituents that would promote lipid bilayer partitioning (hence protecting the drug from hydrolysis) and interfere with the binding of the drug carboxylate form to human serum albumin (hence preventing a shift in the equilibrium favoring the inactive form). See Wall JG, et al. 1992 Anticancer Drugs 3:337-345; Burke T G, et al. 1994, Biochem. 33:10325-10336; Burke TG, et al. 1994, Biochem. 32: 5352-5364. The labs jointly reported the preparation of about two-dozen 7-silylcamptothecins of which many displayed good in vitro activity against multiple cancer cell lines. See Bom D, et al. 2000 J. Med. Chem. 43: 3970-3980. The inherent flexibility of the annulation cascade approach resulted in the synthesis of several hundred more novel silatecans and homosilatecans for biological evaluation. Among the silatecan series, AR-67 (205)-7-tertbutyldimethysilyl-10-hydroxycamptothecin emerged as an impressive candidate for additional in vitro and in vivo evaluation.

As a class, the camptothecins have been studied in numerous solid malignancies. These include non-small cell lung cancer, squamous cell cancer of the head and neck, non-Hodgkin's lymphoma, colorectal cancer, multiple myeloma, and gynecologic tumors. See Cho LC, et al. 2004 Oncology, 13:29-39; Huang C H, et al. 2001 Oncology 61: 14-24; Murphy BA et al. 2007, Oncology 15:47-52; Cabanillas F 1999, Semin. Oncology 36:11-15; Armand J P et al. 1999 Anticancer Drugs, 10: 5-12; Rothenberg ML et al. 1999 Semin Oncology, 26:632-639; Kraut EH et al. 1998 Semin. Hematol. 35:4-32; Verschraegn C F et al. 1998, Ann Acad Med Singapore 27: 683-687. As outlined in Table 1, topotecan (Hycamtin) is FDA approved as a second line therapy for ovarian and small cell lung cancer while irinotecan, (Camptosar) in conjunction with 5-fluorouracil (5-FU), is FDA approved either as a treatment for colon cancer or alone as a salvage therapy for 5-FU refractory colon cancer.

Thus, a need exists for improved methods for treating cancer using camptothecin compounds.

### SUMMARY

The present invention provides methods for treating cancer using a third generation camptothecin compound AR-67, that demonstrates reduced toxicity relative to other camptothecin compounds and demonstrates efficacy when used as a single agent. The invention also provides methods of predicting the response of cancer in a subject to treatment with third generation camtothecin compounds, such as AR-67.

The method for treating cancer may include administering to a patient a composition of a therapeutically effective amount of AR-67, wherein the method is at least as effective as the administration of a dose of other camptothecins delivered as a single agent or as a combination therapy, and wherein the method reduces the incidence of one or more adverse events. The method may advantageously reduce the incidence of one or more adverse events relative to other camptothecins as a single agent or to a combination therapy.

In an aspect, the composition consists essentially of AR-67. In another aspect, the cancer is glioblastoma multiform, myelodysplastic syndrome, metastatic adenocarcinoma, metastatic carcinoma of the colon or rectum, small cell lung cancer, non-small cell lung cancer, soft tissue sarcoma, squamous cell cancer of head and neck, non-Hodgkin's lymphoma, colorectal cancer, bladder cancer, prostate cancer, duodenal cancer, esophageal cancer, pancreatic cancer, multiple myeloma, or gynecologic tumors.

The adverse events may be, e.g., neutropenia, thrombocytopenia, diarrhea, fatigue, leucopenia, and anemia.

In an aspect, the therapeutically effective amount of AR-67 is 1.2-12.4 mg/m²/day or 1.2-7.5 mg/m²/day. The therapeutically effective amount of AR-67 may also be 2.0-11.0 mg/m2/day, 3.0-9.0 mg/m2/day, 4.0-8.0 mg/m2/day, or 5.0-7.0 mg/m2/day. In another aspect, the therapeutically effective amount of AR-67 is 10.0-20.0 mg/m2/day, 15.0-25.0 mg/m2/day, 20.0-30.0 mg/m2/day, or 25.0-35.0 mg/m2/day.

The therapeutically effective amount of AR-67 may be delivered orally or intravenously. In an aspect, the therapeutically effective amount of AR-67 is delivered in the presence or absence of a cremophor.

In an aspect, the patient is dosed with a therapeutically effective amount of AR-67 on days 1-5 of a 21 day cycle. In another aspect, the patient is dosed with a therapeutically effective amount of AR-67 on days 1, 4, 8, 12, and 15, of a 21 day cycle.

The invention may also be a composition for use in a method for treating cancer in a patient, the method comprising any one of the methods of claims 1 to 11.

In an aspect, the therapeutically effective amount of AR-67 is administered as a second-line therapy. In another aspect, the patient had failed treatment with a camptothecin other than AR-67 within 90 days of the administering step. In yet another aspect, the camptothecin other than AR-67 is bevacizumab.

### BRIEF DESCRIPTION OF THE DRAWINGS

FIG. 1 is an illustration of the structure of AR-67.

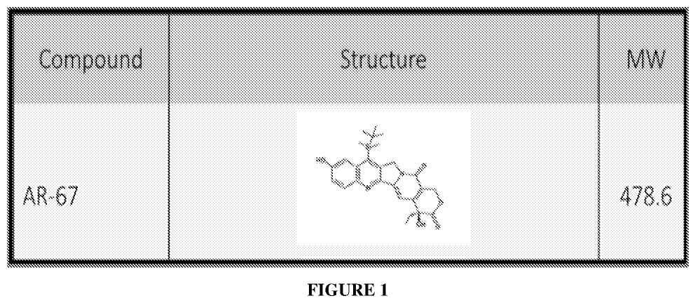

### DETAILED DESCRIPTION OF THE PREFERRED EMBODIMENTS

This disclosure describes, in part, the results of clinical trial Protocol # ARN-AR67-IIS202. The entire results of the protocol may be found at Clinicaltrials.gov, which is incorporated herein by reference in its entirety.

The following examples serve to illustrate certain aspects of the disclosure and are not intended to limit the disclosure. The contents of any references, pending patent applications, and published patents cited throughout this application are incorporated herein by reference in their entirety.

### EXAMPLES

### Objectives:

The primary objective of this open-label, multicenter study was to determine the 6-month progression free survival (PFS) when intravenous (IV) AR-67 was administered in adults with confirmed reGBM who had not recently (>90 days) recurred after treatment with bevacizumab (including patients who had received temozolomide, but not bevacizumab). The primary objective in the rapid bevacizumab failure group (<90 days) was to determine the 2-month PFS.

### Protocol Design:

Forty-six patients with recurrent GBM were enrolled into our Phase 2 trial to evaluate the safety, tolerability and efficacy of AR-67. One patient originally assigned to Cohort 1 succumbed to disease prior to dosing, leaving a total of 45 patients who received at least one dose of drug. The 45 patients were divided into two initial Cohorts:
Cohort 1 consisted of patients (N=30) who either had never received bevacizumab, or had not failed bevacizumab within 90 days or more after their last dose.
Cohort 2 consisted of patients (N=13) who had recently received bevacizumab, but had failed/progressed within 90 days of their last dose despite treatment.
Cohort 3 was designated retrospectively, and consisted of 2 patients whose bevacizumab status was unknown or not captured.

### Dose/Route/Schedule:

AR-67 was administered once daily by 1 hour IV infusion for 5 consecutive days on a 21-day cycle. AR-67 was supplied in sterile 3 ml vials, containing 1 mg of AR-67 (5 mg/mL) in cremophor/ethanol diluent for IV infusion. This solution was reconstituted in dextrose 5% in water to a final concentration between 0.06-0.6 mg/mL Drug (7.5 mg/m2) was to be given until the onset of toxicity requiring discontinuation or tumor progression. Tumor response was assessed=14d after every second cycle and before every third cycle using MRI.

### Toxicity Summary

AR-67 does not display the severe dose limiting toxicities typical of the marketed standard, Irinotecan.

| Title of the Study: | A Phase 2 Study of AR-67 (7-t-butyldimethylsiltyl-10- | | |
|---|---|---|---|
| | hydroxycamptothecin) in Adult Patients with Recurrence of | | |
| | Glioblastoma Multiforme (GBM) or Gliosarcoma | | |
| Investigator(s)/ | Multi-center | | |
| Study Centre(s): | | | |
| Publication(s): | Not applicable | | |
| Studied Period: | 11 Dec. 2009 (First patient's informed | Clinical | 2 |

| | |
|---|---|
| consent date) to 8 Apr. 2014 (Last | Phase: |
| patient's last visit or contact) | |

Objectives:
The primary objective of this study was to determine the 6-month progression free survival (PFS)
when intravenous (IV) AR-67 was administered in adults with confirmed recurrence of
glioblastoma multiforme (GBM) who had not recently (>90 days) recurred after treatment with
bevacizumab (including patients who had received temozolomide, but not bevacizumab). The
primary objective in the rapid bevacizumab failure group (<90 days) was to determine the 2-
month PFS.
Secondary objectives were to determine the following when AR-67 was administered to
patients with recurrent GBM:
The effect of AR-67 on overall survival (OS)
The effect of AR-67 on overall PFS
The effect of AR-67 on median event-free survival (EFS)
The impact of AR-67 on tumor response in patients with measurable disease
The safety and tolerability of AR-67
Methodology:
This was a Phase 2, open-label study designed to evaluate the efficacy of IV AR-67
administered once daily for 5 days on a 21-day cycle to adults with recurrence of GBM.
There were 2 cohorts. The first cohort consisted of patients who had not received or had not
recently failed (>90 days) bevacizumab. The second cohort consisted of patients who had failed
bevacizumab within the 90 days prior to screening.
The starting dose of AR-67 was 7.5 mg/m². AR-67 was to be given until the onset of toxicity
requiring discontinuation or tumor progression. Patients with toxicities of AR-67 warranting
dose adjustments were allowed guideline-specified dose level reductions. Treatment with AR-
67 was to be discontinued if toxicities required more than 2 dose reductions or a reduction
beyond dose level -2. AR-67 was to be given until the onset of toxicity requiring
discontinuation or tumor progression.
Tumor response was to be assessed at least 14 days after every second cycle of treatment, prior
to the start of every third cycle, using contrast and non-contrast brain magnetic resonance
imaging (MRI) with assessment based on the MacDonald criteria. All efforts were made to
maintain the patient on the same AR-67 dose until the radiographic tumor measurement was
done after completion of Cycle 2. Patients with progressive disease were to be discontinued and
receive off-protocol treatment.
Safety assessments included adequate monitoring of patients by investigators and/or designated
clinical staff consistent with the schedule of assessments and procedures, and reporting of all
toxicities and potential toxic events.
Number of Patients:
Thirty-two (32) patients were planned to be accrued to the non-recent bevacizumab failure
cohort and 26 patients were planned to be accrued to the bevacizumab failure cohort, for a
total of 58 patients.
Due to early study termination, 46 patients were enrolled: 31 patients in the non-recent
bevacizumab failure cohort (Cohort 1), 13 patients in the bevacizumab failure cohort (Cohort
2), and 2 additional patients without reported bevacizumab history. Of these 46 patients, 45
patients received at least one dose of AR-67 and were analyzed for safety.
Stable disease was the best overall disease response observed in 7/46 patients (5
patients in Cohort 1 and 2 patients in Cohort 2).
The disease status of the majority of patients at study completion/discontinuation, and the
best overall response, was progressive disease.
Safety:
AR-67 was well tolerated.
There were 4 deaths in this study; one death occurred during screening and prior to study
drug administration, and 3 occurred following drug administration that were due to disease
progression.
A total of 17 (38%) patients experienced serious adverse events (SAEs). The most common
SAE was headache which occurred in 3 patients.
The rate of withdrawals due to TEAEs was low with only 1 patient documented as
discontinuing due to an AE on the Study Completion CRF, though TEAEs leading to
discontinuation were reported in 9 (20.0%) patients.
The majority of patients reported at least 1 TEAE (91%) and at least 1 treatment-related
TEAE (82%). Most TEAEs were mild or moderate in intensity and Grade 1-3 in toxicity.
For patients who had received or not recently failed (>90 days) treatment with
bevacizumab (Cohort 1), the most common TEAEs were fatigue (27%), constipation
(23%), headache (23%), anaemia (23%), and thrombocytopenia (20%).
For patients who had recently (<90 days) failed treatment with bevacizumab (Cohort 2),
the most common TEAEs were nausea (31%), constipation (15%), diarrhoea (15%),
fatigue (15%), muscular weakness (15%), confusional state (15.%), headache (15%), and
hiccups (15%).
There were a number of patients with TEAEs due to laboratory abnormalities including
events from the following system organ classes: blood and lymphatic system disorder (17
[38%] patients), investigations (15 [33%] patients), and metabolism and nutrition
disorders (11 [24%] patients). Three TEAEs associated with laboratory abnormalities
were assessed as Grade 4 in toxicity.
Overall, laboratory values were similar between the 2 groups of patients and were within
acceptable ranges.
CONCLUSIONS:
AR-67 was well tolerated and exhibited a safety profile consistent with the camptothecin class
of drugs.
While the majority of treated patients did not complete the study due to disease
progression, partial response was the best overall response observed in 3/46 patients and
stable disease was the best overall response observed in 7/46 patients.

Table 2 presents a summary of patient disposition and reasons for withdrawal from the study and Table 3 presents a summary of the disease status of patients in the study.

TABLE 2
Summary of Patient Disposition and Reasons for Withdrawal (All Patients)

| | Cohort 1 | Cohort 2 | All Patients |
|---|---|---|---|
| | (N = 31) | (N = 13) | (N = 46)¹ |
| Enrolled | 31 (100) | 13 (100) | 46 (100) |
| Safety population² | 30 (96.8) | 13 (100) | 45 (97.8) |
| Completed all study visits | 4 (12.9) | 2 (15.4) | 6 (13.0) |
| Early discontinuation from study | 26 (83.9) | 10 (76.9) | 37 (80.4) |
| Disease progression | 24 (77.4) | 7 (53.8) | 32 (69.6) |
| Death | 2 (6.5) | 1 (7.7) | 3 (6.5) |
| Adverse event | 0 | 1 (7.7) | 1 (2.2) |
| Treatment delay >21 days | 0 | 1 (7.7) | 1 (2.2) |
| Patients who left the study | 1 (3.3) | 1 (7.7) | 3 (6.5) |
| for unknown reason | | | |

Cohort 1 = patients who had not received or not recently failed (>90 days) bevacizumab. Cohort 2 = patients who had recently (<90 days) failed bevacizumab.
¹Two patients, #03-01 and #03-32, did not provide bevacizumab treatment history and are not included in either cohort, but are included in the All Patients data.
² The Safety Population included enrolled patients who took at least one dose of study drug.

TABLE 3
Summary of Disease Status (All Patients)

| | Cohort 1 | Cohort 2 | All Patients 1 |
|---|---|---|---|
| Disease Status | (N = 31) | (N = 13) | (N = 46) |
| Best Overall Response | | | |
| Complete Response | 0 | 0 | 0 |
| Partial Response | 3 | 0 | 3 |
| Stable Disease | 5 | 2 | 7 |
| Progressive Disease | 20 | 10 | 31 |
| Missing | 3 | 1 | 5 |
| Response at Study | | | |
| Completion or | | | |
| Discontinuation | | | |
| Complete Response | 0 | 0 | 0 |
| Partial Response | 1 | 0 | 1 |
| Stable Disease | 0 | 2 | 2 |
| Progressive Disease | 28 | 10 | 39 |
| Missing | 2 | 1 | 4 |

Cohort 1 = patients who had not received or not recently failed (>90 days) bevacizumab.
Cohort 2 = patients who had recently (<90 days) failed bevacizumab.
¹ Two patients, #03-01 and #03-32, did not provide bevacizumab treatment history and are not
included in either cohort, but are included in the All Patients data.

### Efficacy Conclusions

Partial response was the best overall response observed in 3/46 patients, all in Cohort 1. One patient had durable partial response at study completion, following 14 cycles of treatment with AR-67.

Stable disease was the best overall response observed in 7/46 patients (5 patients in Cohort 1 and 2 patients in Cohort 2).

The disease status of the majority of patients at study completion/discontinuation, and the best overall response, was progressive disease.

### Brief Summary of Adverse Events

An overview of treatment emergent adverse events (TEAEs) reported in this study is presented in Table 4. There were 4 deaths in this study, one prior to any study drug treatment, and 3 due to disease progression. Two deaths due to disease progression were reported as TEAEs with fatal outcome (i.e., TEAEs leading to death).

A total of 17 (37.8%) patients experienced at least one serious adverse event (SAE). There were 30 SAEs in total with 9 (30.0%) patients in Cohort 1 (non-recent bevacizumab failures) reporting 16 events and 8 (61.5%) patients in Cohort 2 (recent bevacizumab failures) reporting 14 events. Approximately half the SAEs (19/29) were related to treatment with AR-67 and were reported in 11 patients.

TEAEs that led to early discontinuation were reported in 5 (16.7%) patients in Cohort 1 and 4 (30.8%) patients in Cohort 2. However, only 1 patient was recorded as having discontinued the study due to an AE on the Study Completion case report form. The majority of patients had a TEAE (91.1%) and a treatment-related TEAE (82.2%). There were 428 TEAEs, with 346 events in 27 (90.0%) patients in Cohort 1 and 55 events in 12 (92.3%) patients in Cohort 2. Overall, the most common TEAE was fatigue which occurred in 24.4% of all patients. Shown in Table 5 is a summary of Treatment Emergent Adverse Events (TEAEs) with an incident rate 10%. In Cohort 1, the most common TEAEs were fatigue (26.7%), constipation (23.3%), headache (23.3%), anaemia (23.3%), and thrombocytopenia (20.0%). In Cohort 2, the most common TEAEs were nausea (30.8%), constipation (15.4%), diarrhoea (15.4%), fatigue (15.4%), muscular weakness (15.4%), confusional state (15.4%), headache (15.4%), and hiccups (15.4%).

The majority of events were mild or moderate in intensity and Grade 1-3 in severity (based on the National Cancer Institute—Common Terminology Criteria for Adverse Events [NCI-CTCAE] grading; Table 7). There were 4 events in 4 patients documented with life-threatening intensity, but none documented as NCI-CTCAE Grade 5. There were also 9 events documented as NCI-CTCAE Grade 4.

TABLE 4
Overview of Treatment Emergent Adverse Events
(Safety Population)

| | Cohort 1 | | Cohort 2 | | All Patients¹ | |
|---|---|---|---|---|---|---|
| | (N = 30) | | (N = 13) | | (N = 45) | |
| | | No of | | No of | | No of |
| | n (%) | Events | n (%) | Events | n (%) | Events |
| ≥1 TEAE² | 27 | 346 | 12 | 55 | 41 | 428 |
| | (90.0) | | (92.3) | | (91.1) | |
| ≥1 related | 25 | 258 | 10 | 28 | 37 | 304 |
| TEAE³ | (83.3) | | (76.9) | | (82.2) | |
| Death | 1 | 1 | 2 | 2 | 3 | 3 |
| | (3.3) | | (15.4) | | (6.7) | |
| ≥1 SAE | 9 | 16 | 8 | 14 | 17 | 30 |
| | (30.0) | | (61.5) | | (37.8) | |
| 1 related | 6 | 10 | 5 | 9 | 11 | 19 |
| SAE³ | (20.0) | | (38.5) | | (24.4) | |
| ≥1 TEAE | 5 | 5 | 4 | 5 | 9 | 10 |
| leading | (16.7) | | (30.8) | | (20.0) | |
| to early | | | | | | |
| discontin- | | | | | | |
| uation | | | | | | |
| ≥1 TEAE | 1 | 16 | 1 | 1 | 2 | 17 |
| leading | (3.3) | | (7.7) | | (4.4) | |
| to_death | | | | | | |

Abbreviations: SAE = serious adverse event; TEAE = treatment emergent adverse event.
% = 100 × n/N, where n = # of patients in the specified category and N = # of patients in the Safety Population.
Cohort 1 = patients who had not received or not recently failed (>90 days) bevacizumab.
Cohort 2 = patients who had recently (<90 days) failed bevacizumab.
¹Two patients, #03-01 and #03-32, did not provide bevacizumab treatment history and are not included in either cohort, but are included in the All Patients data.
² TEAEs were adverse events with date of onset that occurred on or after the date of first dose of study drug.
³ Related = possibly, probably, or definitely related.

TABLE 5
Summary of Treatment Emergent Adverse Events With
an Incidence Rate of >10% in Either
Patient Cohort, by System Organ Class (Safety Population)

| | Cohort 1 | Cohort 2 | All Patients¹ |
|---|---|---|---|
| System Organ Class | (N = 30) | (N = 13) | (N = 45) |
| Preferred Term | n (%) | n (%) | n (%) |
| Any TEAE | 27 | 12 | 41 |
| | (90.0) | (92.3) | (91.1) |
| Gastrointestinal Disorders | 15 | 7 (53.8) | 23 |
| | (50.0) | | (51.1) |
| Constipation | 7 (23.3) | 2(15.4) | 9 (20.0) |
| Nausea | 4(13.3) | 4 (30.8) | 9 (20.0) |
| Diarrhoea | 5(16.7) | 2(15.4) | 7(15.6) |
| Dyspepsia | 4(13.3) | 0 | 4 (8.9) |
| Vomiting | 3 (10.0) | 1 (7.7) | 4 (8.9) |
| Nervous System Disorders | 15 | 6 (46.2) | 23 |
| | (50.0) | | (51.1) |
| Headache | 7 (23.3) | 2(15.4) | 10 |
| | | | (22.2) |
| Dizziness | 5(16.7) | 0 | 5(11.1) |
| Convulsion | 3 (10.0) | 1 (7.7) | 4 (8.9) |
| Blood and Lymphatic | 14 | 2 (15.4) | 17 |
| System Disorders | (46.7) | | (37.8) |
| Thrombocytopenia | 6 (20.0) | 1 (7.7) | 8(17.8) |
| Anaemia | 7 (23.3) | 0 | 7(15.6) |
| Neutropenia | 5 (16.7) | 1 (7.7) | 6(13.3) |
| Leukopenia | 5 (16.7) | 0 | 5(11.1) |
| General Disorders and Administration Site | 11 | 5 (38.5) | 17 |
| Conditions | (36.7) | | (37.8) |
| Fatigue | 8 (26.7) | 2(15.4) | 11 |
| | | | (24.4) |
| Investigations | 12 | 1 (7.7) | 15 |
| | (40.0) | | (33.3) |
| Neutrophil count decreased | 5 (16.7) | 0 | 6(13.3) |
| Lymphocyte count decreased | 5 (16.7) | 0 | 5(11.1) |
| White blood cell count decreased | 5 (16.7) | 0 | 5(11.1) |
| Platelet count decreased | 4(13.3) | 0 | 4 (8.9) |
| Musculoskeletal and Connective Tissue | 10 | 3 (23.1) | 13 |
| Disorders | (33.3) | | (28.9) |
| Muscular weakness | 3 (10.0) | 2(15.4) | 5(11.1) |
| Musculoskeletal pain | 3 (10.0) | 0 | 3 (6.7) |
| Psychiatric Disorders | 9 (30.0) | 3 (23.1) | 13 |
| | | | (28.9) |
| Confusional state | 3 (10.0) | 2(15.4) | 5(11.1) |
| Metabolism and Nutrition Disorders | 8 | 2 | 11 |
| | (26.7) | (15.4) | (24.4) |
| Hyperglycaemia | 4 | 0 | 4 (8.9) |
| | (13.3) | | |
| Hypopho sphataemia | 4 | 0 | 4 (8.9) |
| | (13.3) | | |
| Respiratory, Thoracic, and Mediastinal | 9 | 2 | 11 |
| Disorders | (30.0) | (15.4) | (24.4) |
| Hiccups | 3 | 2 | 5(11.1) |
| | (10.0) | (15.4) | |
| Vascular Disorders | 7 | 0 | 8 (17.8) |
| | (23.3) | | |
| Flushing | 5 | 0 | 5(11.1) |
| | (16.7) | | |
| Eye Disorders | 3 | 1 (7.7) | 4 (8.9) |
| | (10.0) | | |
| Vision blurred | 3 | 1 (7.7) | 4 (8.9) |
| | (10.0) | | |

Abbreviations:
TEAE = treatment emergent adverse event.
% = 100 × n/N, where n = # of patients in the Preferred Term category and N = # of patients in the Safety Population. Adverse events were coded using MedDRA version15.1.
Cohort 1 = patients who had not received or not recently failed (>90 days) bevacizumab.
Cohort 2 = patients who had recently (<90 days) failed bevacizumab.
¹Two patients, #03-01 and #03-32, did not provide bevacizumab treatment history and are not included in either cohort, but are included in the All Patients data.

### Analysis of Adverse Events Overall

Approximately 91% of all patients experienced a TEAE, including 27 (90.0%) patients in Cohort 1, 12 (92.3%) patients in Cohort 2, and Patients #03-01 and #03-32 who were in neither cohort. Overall, the most common TEAEs were associated with the following system organ classes (SOCs): gastrointestinal disorders, nervous system disorders, blood and lymphatic system disorders, and general disorders and administration site conditions (Table 5). The most common TEAEs in Cohort 1 were fatigue (26.7%), constipation (23.3%), headache (23.3%), anaemia (23.3%), and thrombocytopenia (20.0%). The most common TEAEs in Cohort 2 were nausea (30.8%), constipation (15.4%), diarrhoea (15.4%), fatigue (15.4%), muscular weakness (15.4%), confusional state (15.4%), headache (15.4%), and hiccups (15.4%). There were less instances of patients with blood and lymphatic system disorder TEAEs in Cohort 2 (3 events in 2 [15.4%] patients) compared to Cohort 1 (41 events in 14 [46.7%] patients) or with events in the investigations SOC (2 events in 1 [7.7%] patient in Cohort 2 vs. 101 events in 12 [40%] patients in Cohort 1). Additionally, there were no patients in Cohort 2 reported to have a decreased neutrophil count, lymphocyte count, white blood cell (WBC) count, and platelet count compared to 4-5 patients in Cohort 1.

### Analysis of Adverse Events by Relationship to Study Treatment

A summary of TEAEs by relationship to AR-67 is provided in Table 6.

The majority of patients experienced TEAEs that were either not related (29 [64.4%] patients) or possibly related to study treatment (28 [62.2%] patients). TEAEs that were probably related or definitely related were reported in 15 (33.3%) patients and 19 (42.2%) patients, respectively. The incidence of related TEAEs were similar in Cohort 1 and Cohort 2 with the exception that there was only 1 patient in Cohort 2 who experienced an AE that was probably related to AR-67 (AE preferred term=cellulitis), compared to 12 patients in Cohort 1.

Overall, the most common treatment-related (i.e., possibly, probably, or definitely related) TEAE was fatigue. In patients who had not recently failed bevacizumab treatment (Cohort 1), the most common treatment related TEAEs were those associated with blood and lymphatic system disorders (i.e., anaemia, thrombocytopenia, neutropenia, leukopenia) and investigations (i.e., decreased leukocytes, decreased neutrophils, and decreased WBCs), as well as fatigue and constipation. In patients who had recently failed bevacizumab treatment (Cohort 2), the most common treatment-related TEAEs were nausea, fatigue, and muscular weakness.

TABLE 6
Treatment Emergent Adverse Events by Relationship to
AR-67 (Safety Population)

| | Cohort 1 | | Cohort 2 | | All Patients¹ | |
|---|---|---|---|---|---|---|
| | (N = 30) | | (N = 13) | | (N = 45) | |
| | | No of | | No of | | No of |
| | n (%) | Events | n (%) | Events | n (%) | Events |
| Total | 27 | 346 | 12 | 55 | 41 | 428 |
| | (90.0) | | (92.3) | | (91.1) | |
| Not Related | 20 | 83 | 7 | 25 | 29 | 117 |
| | (66.7) | | (53.8) | | (64.4) | |
| Possibly | 20 | 146 | 9 | 19 | 28 | 170 |
| Related | (66.7) | | (53.8) | | (62.2) | |
| Probably | 12 | 76 | 1 | 1 | 15 | 79 |
| Related | (40.0) | | (7.7) | | (33.3) | |
| Definitely | 12 | 36 | 5 | 8 | 19 | 55 |
| Related | (40.0) | | (38.5) | | (42.2) | |

% = 100 × n/N, where n = # of patients in the category and N = # of patients in the Safety Population.
Cohort 1 = patients who had not received or not recently failed (>90 days) bevacizumab.
Cohort 2 = patients who had recently (<90 days) failed bevacizumab.
Discrepancies between total number of events and sum of events presented by relationship to AR-67 are due to missing investigator assessment of relationship to AR-67.
¹Two patients, #03-01 and #03-32, did not provide bevacizumab treatment history and are not included in either cohort, but are included in the All Patients data.

### Analysis of Adverse Events by Severity and Toxicity Grade

A summary of TEAEs by severity (intensity) and toxicity grade is provided in Table 7.

Overall, more than half the Safety Population experienced AEs that were documented as mild, moderate, and severe intensity (between 58-69% patients). The majority of events were mild (212/428) or moderate (128/428) in intensity. There were 4 events in 4 patients that were considered life-threatening events. In Cohort 1 (non-recent bevacizumab failures), there were 3 events of life-threatening intensity and each experienced by 1 patient. These events were anaemia, pleural effusion, and platelets. In Cohort 2 (recent bevacizumab failures), one patient had an event of life-threatening intensity, an event of intracranial venous sinus thrombosis. Pleural effusion and intracranial venous sinus thrombosis were reported as SAEs. The most common severe events in Cohort 1 were neutropenia (5 [16.7%]), neutrophil count decreased (3 [10.0%] patients), and WBC count decreased (3[10.0%] patients); all other severe events occurred in 1 or 2 patients. Two patients had severe AEs of headache and all other severe events occurred in 1 patient.

The majority of events were Grade 1-3 in toxicity based on the NCI-CTCAE grading system, with only 9 Grade 4 events and no documented Grade 5 events. In Cohort 1, there were 5 events assessed as NCI— CTCAE Grade 4. These events were neutrophil count decreased, blood glucose increased, pleural effusion (life-threatening intensity), and the uncoded events of cardiac ischemia/infarction and platelets. In Cohort 2, there were 3 events assessed as NCI-CTCAE Grade 4, pancytopenia, muscular weakness, and intracranial venous sinus thrombosis (life-threatening intensity).

A summary of TEAEs by severity is provided in Table 7 by intensity and by NCI-CTCAE toxicity grade.

TABLE 7
Treatment Emergent Adverse Events by Severity (Intensity) and
NCI-CTCAE Toxicity Grade (Safety Population)

| | Cohort 1 | | Cohort 2 | | All Patients¹ | |
|---|---|---|---|---|---|---|
| | (N = 30) | | (N = 13) | | (N = 45) | |
| | | No of | | No of | | No of |
| | n (%) | Events | n(%) | Events | n (%) | Events |
| Total | 27 | 346 | 12 | 55 | 41 | 428 |
| | (90.0) | | (92.3) | | (91.1) | |
| Intensity² | | | | | | |
| Mild | 22 | 177 | 7 | 20 | 31 | 212 |
| | (73.3) | | (53.8) | | (68.9) | |
| Moderate | 20 | 100 | 4 | 18 | 26 | 128 |
| | (66.7) | | (30.8) | | (57.8) | |
| Severe | 18 | 59 | 8 | 14 | 28 | 75 |
| | (60.0) | | (61.5) | | (62.2) | |
| Life Threatening | 3 | 3 | 1 | 1 | 4 | 4 |
| | (10.0) | | (7.7) | | (8.9) | |
| NCI-CTCAE | | | | | | |
| Grade² | | | | | | |
| Grade 1 | 21 | 178 | 7 | 17 | 30 | 209 |
| | (70.0) | | (53.8) | | (66.7) | |
| Grade 2 | 18 | 94 | 4 | 16 | 23 | 119 |
| | (60.0) | | (30.8) | | (51.1) | |
| Grade 3 | 19 | 63 | 9 | 16 | 30 | 82 |
| | (63.3) | | (69.2) | | (66.7) | |
| Grade 4 | 5 | 5 | 3 | 3 | 9 | 9 |
| | (16.7) | | (23.1) | | (20.0) | |
| Grade 5 | 0 | 0 | 0 | 0 | 0 | 0 |

Abbreviations: NCI-CTCAE = National Cancer Institute Common Terminology Criteria for Adverse Events.
% = 100 × n/N, where n = # of patients in the category and N = # of patients in the Safety Population.
Cohort 1 = patients who had not received or not recently failed (>90 days) bevacizumab.
Cohort 2 = patients who had recently (<90 days) failed bevacizumab.
¹Two patients, #03-01 and #03-32, did not provide bevacizumab treatment history and are not included in either cohort, but are included in the All Patients data.
²Only the highest grade is counted for multiple occurrences of the same TEAE in one individual. Discrepancies between total number of TEAEs and sum of TEAEs presented by severity (intensity) or by NCI-CTCAE toxicity grade are due to missing investigator assessment of severity or grade.

### Other Serious Adverse Events

There were 30 treatment-emergent SAEs in this study experienced by 17 patients including 16 SAEs in 9 patients in Cohort 1 and 14 SAEs in 8 patients in Cohort 2. There was a higher incidence of SAEs in Cohort 2 (61.5%) compared to Cohort 1 (30%), with a similar number of events in each cohort.

Three patients had their treatment with AR-67 permanently discontinued due to an SAE, but “Adverse Event” was only listed as the reason for withdrawal for one patient, #04-08, while the other 2 patients (04-13 and #05-39) were reported to have discontinued due to disease progression. Six patients had their treatment with AR-67 temporarily interrupted or stopped due to an SAE and a dose modification due to an SAE was reported in 2 patients. Over half the SAEs (19/30 events) were determined to be related (possibly, probably, or definitely related) to study treatment.

The most common SAE overall was headache, which occurred in 3 patients (1 patient in Cohort 1 and 2 patients in Cohort 2). In Cohort 1, the most common SOCs were infections and infestations (4 events in 3 patients), nervous system disorders (3 events in 3 patients), and respiratory, thoracic and mediastinal disorders (3 events in 2 patients). In Cohort 2, the most common SOCs were nervous system disorders (6 events in 6 patients), and blood and lymphatic system disorders (3 events in 2 patients).

### Other Significant Adverse Events

There were 9 patients with 10 TEAEs leading to early discontinuation; 5 patients in Cohort 1 and 4 patients in Cohort 2. Only one patient, #04-08, was documented as discontinuing the study due to an AE on the Study Completion CRF. One patient, #07-18, died due to disease progression and 2 patients, #06-12 and #08-45, had AEs documented as leading to discontinuation; however their Study Completion CRF indicated they had completed the study. The other 5 patients were discontinued due to disease progression, including Patient #05-39 who experienced SAEs which were reported to have led to permanent discontinuation of treatment with AR-67. Six of the 10 TEAEs leading to discontinuation were from the nervous system disorders SOC. The most common TEAE leading to discontinuation was headache (3 patients) and the other TEAEs leading to discontinuation all occurred in 1 patient each.

### Safety Conclusions

AR-67 was well tolerated.

There were 4 deaths in this study; one death occurred during screening and prior to study drug administration, and 3 occurred following drug administration that were due to disease progression.

A total of 17 (38%) patients experienced SAEs. The most common SAE was headache which occurred in 3 patients.

The rate of withdrawals due to TEAEs was low with only 1 patient documented as discontinuing due to an AE on the Study Completion CRF, though TEAEs leading to discontinuation were reported in 9 (20.0%) patients.

The majority of patients reported at least 1 TEAE (91%) and at least 1 treatment-related TEAE (82%). Most TEAEs were mild or moderate in intensity and Grade 1-3 in toxicity.

For patients who had not received or not recently failed (>90 days) treatment with bevacizumab (Cohort 1), the most common TEAEs were fatigue (27%), constipation (23%), headache (23%), anaemia (23%), and thrombocytopenia (20%).

For patients who had recently (<90 days) failed treatment with bevacizumab (Cohort 2), the most common TEAEs were nausea (31%), constipation (15%), diarrhoea (15%), fatigue (15%), muscular weakness (15%), confusional state (15%), headache (15%), and hiccups (15%).

There were a number of patients with TEAEs due to laboratory abnormalities including events from the following SOCs: blood and lymphatic system disorder (17 [38%] patients), investigations (15 [33%] patients), and metabolism and nutrition disorders (11 [24%] patients). Three TEAEs associated with laboratory abnormalities were assessed as Grade 4 in toxicity.

Overall, laboratory values were similar between the 2 groups of patients and were within acceptable ranges.

### DISCUSSION

Despite advances in therapeutic interventions, individuals diagnosed with GBM have a poor prognosis. These individuals have a 12-15 month median survival following their initial diagnosis of GBM and, for those who achieve remission, a 3-9 month median survival with recurrent GBM. Additionally, there are fewer treatment options available for individuals with recurrent GBM. AR-67 is a third generation camptothecin, a potent class of anticancer drugs that inhibit DNA topoisomerase I. AR-67 has been shown to have significant tumor uptake and retention in preclinical studies, and was selected by the National Cancer Institute for development in the first Cycle of the Rapid Access to Intervention Department (RAID) program. This Phase 2 study was designed to explore the efficacy of AR-67 in patients with recurrent GBM and to evaluate whether a definitive proof of concept study should be pursued in this disease setting with AR-67. Efficacy was to be based on 6 month PFS in patients who had not recently failed treatment with bevacizumab (Cohort 1) or 2 month PFS in patients who had recently failed treatment with bevacizumab (Cohort 2). Due to lack of efficacy and after concluding that no further benefits would be obtained, the Sponsor decided to terminate the study.

There were 4 deaths in this study, however 3 deaths were related to disease progression and were not related or unlikely related to study treatment, while the fourth occurred during screening prior to AR-67 administration. The majority of patients were discontinued from the study due to disease progression, with the population's general weakened health likely contributing to the number of TEAEs observed in this study.

Approximately 91% of all patients experienced a TEAE and 82% experienced a treatment related TEAE, with the majority of events being mild or moderate in severity and toxicity (NCI-CTCAE Grades 1-3). The most common TEAE overall was fatigue. The TEAEs that occurred in approximately >10% of all patients were expected events and toxicities for AR-67 and other camptothecins, such as those associated with gastrointestinal disorders, nervous system disorders, and blood and lymphatic system disorders.

The toxicities associated with AR-67 included hematological AEs, typical of myelosuppressive treatments. In this study, blood and lymphatic systems disorders were observed in approximately 40% of the population with thrombocytopenia, anaemia, neutropenia, and leukopenia occurring in 11-18% of all patients. These results were comparable to those observed in recent Phase I and II trials with camptothecin analogs, karenitecin and gimatecan, which are also being evaluated as treatments for GBM. In a Phase II trial with oral gimatecan, treatment-related toxicities of thrombocytopenia, leukopenia, and neutropenia were observed in 10-18% of patients while a Phase I trial with karenitecin observed neutropenia and thrombocytopenia were observed in 13-23% of patients. See Hu, J et al., 2013, J. Neurooncol. 111:347-353; Grossman, S A et al., 2008 Neuro Oncol 10:608-616.

A common new treatment for recurrent GBM is bevacizumab, a humanized monoclonal antibody against vascular endothelial growth factor with a long terminal half-life. See Avastin (bevacizumab) Product Monograph. Hoffmann-La Roche Limited. Submission Control No. 184140. Date of Authorization: Aug. 21, 2015. GBM patients who fail this line of therapy have not found any benefit from other therapies. This study was designed to evaluate benefits to patients who had recently (<90 days) failed bevacizumab treatment in addition to those who had not.

Overall, there were 30 SAEs in this study, reported in 17 (38%) patients, with less than half of the events (11/30) related to study treatment. There was a higher incidence of SAEs observed in patients who had recently failed bevacizumab (Cohort 2; 60%) compared to those who had not (Cohort 1; 30%), however the number of events and related events were similar between the 2 groups. Interestingly, there were less TEAEs associated with blood and lymphatic system disorders observed in the recent bevacizumab failure cohort (15 vs. 47%) as well as fewer laboratory investigations. This could be due to patients in the recent bevacizumab failure cohort being in the study for no more than 2 cycles of treatment, however, the overall incidences of TEAEs and related TEAEs were similar between the 2 cohorts. However, in a Phase II trial with bevacizumab in combination with irinotecan, a second generation camptothecin, there were also no patients with a Grade 4 hematologic toxicity or Grade 3 and above nonhematologic toxicity.

In this study, AR-67 was shown to be generally well tolerated and exhibited an overall safety profile that was consistent with the camptothecin class of drugs.

### DISCUSSION/CONCLUSIONS

AR-67 was designed to circumvent the major limitations of camptothecin-based chemotherapies which, otherwise, constitute a widely used drug class for treating solid tumors. These limitations include dose limiting toxicities and modest progression free survival rates (PFS6 consistently<20%) when used as a single agent in GBM. See Vredenburgh, J J et al., 2007 Clin Cancer Res. 13:1253-1259. Here, AR-67 was evaluated as a single agent for activity and safety in a Phase 2 trial with patients who were diagnosed with recurrent GBM, a highly aggressive cancer with poor survival rates.

Our Findings Show:
20% of rGBM patients who had never taken bevacizumab, or had not failed bev treatment within 90 days of last dose, surpassed the PFS6 endpoint; suggesting that AR-67 should be evaluated in a larger trial in this patient population.
rGBM patients who failed bevacizumab within 90 days of last dose fared poorly, suggesting they were further along in the disease process, or that they constituted a different sub-population of GBM;
AR-67's safety profile suggests a significant improvement in the major dose-limiting toxicities of this otherwise powerful chemotherapeutic drug class.

Table 8 presents a comparison of the toxicity profiles of AR-67 from this study as a single agent used in a recurrent glioblastoma (reGBM) trial, and Irinotecan, a second generation camptothecin, used in 5 separate colorectal cancer trials.

TABLE 8
AR-67 Phase II Study Toxicity Compared to Irinotecan Toxicity

| | | Irinotecan | | | |
|---|---|---|---|---|---|
| | AR-67 | | | Metastatic | |
| | reGBM | Colorectal | Colorectal | Colorectal | |
| | Single agent, | Single agent, | w/5FU, LV; | Previously | Recurrent Colorectal |
| | infusion | bolus | bolus | treated | N = 316 across |
| | N = 45 | N = 223 | N = 225 | N = 304 | 2 studies |
| Neutropenia | 0% | 12% | 24% | 12% | 18% |
| Grade 4 | | | | | |
| Diarrhoea | 0% | 7% | 5% | 14%** | 22%* |
| Grade 4 | | | | | |
| Nausea | 2% | 16% | 16% | 17% | 12%* |
| Grades 3,4 | | | | | |
| Vomiting | 0% | 12% | 10% | 12% | 14%* |
| Grades 3,4 | | | | | |

*= average across 2 studies
**= late diarrhoea

This comparison of the toxicities seen with AR-67 treatment to Irinotecan treatment shows that AR-67 treatment results in dramatically reduced toxicities relative to the toxicities induced by Irinotecan treatment.

Table 9 presents a comparison of the toxicity profiles of AR-67 from this study as a single agent in a reGBM trial, and Etirinotecan, a pegylated long acting Irinotecan, in either a GBM/AA trial or a BCBM trial. See Nagpal S, et al. 2015, J. Neurooncol. 123:277-282; Cortes J, et al. 2017 Breast Cancer Res. Treat. 165:329-341.

TABLE 9
AR-67 Phase II Study Toxicity Compared to Etirinotecan Toxicity

| | | Etirinotecan | |
|---|---|---|---|
| | AR-67 | GBM/AA | |
| | reGBM | Single agent Phase II, | BCBM |
| | Single agent | N = 20 in heavily | Phase 3: BEACON |
| | N = 45 | pretreated bev failures | Study N = 425 |
| Neutropenia | 13% | NR | 10% |
| Grades 3-4 | All grade 3 | | |
| Diarrhoea | 0% | 10% | 10% |
| Grades 3-5 | | | |
| Nausea | 2% | NR | 4% |
| Grades 3-5 | | | |
| Vomiting | 0% | NR | NR |
| Grades 3-5 | | | |

This comparison of the toxicities seen with AR-67 treatment to Etirinotecan treatment shows that AR-67 treatment has reduced toxicities relative to the toxicities induced by Etirinotecan treatment.

Table 10 presents a comparison of the toxicity profiles of AR-67 from this study as a single agent in a reGBM trial, and Karenitecin, a third generation camptothecin, also in a reGBM trial. See Grossman, S A et al. 2008 Neuro Oncol 10:608-616.

TABLE 10
AR-67 Phase II Study Toxicity Compared to Karenitecin Toxicity

| | AR-67 | Karenitecin |
|---|---|---|
| | reGBM | reGBM |
| | Single agent | Single agent |
| | N = 45 | N = 30 |
| Neutropenia | 13% | 23% |
| Grades 3-4 | All Grade 3 | |
| Thrombocytopenia | 4% | 13% |
| Grades 3-4 | | |
| Diarrhoea | 0% | 3% |
| Grades 3-5 | | |

This comparison of the toxicities seen with AR-67 treatment to Karenitecin treatment shows that AR-67 treatment has reduced toxicities relative to the toxicities induced by Karenitecin treatment.

Table 11 presents a comparison of the toxicity profiles of AR-67 from this study as a single agent in a reGBM trial, and Gimatecan, a third generation camptothecin, also in reGBM trials in two different doses. See Hu, Jet al. 2013 J. Neurooncol. 111:347-353.

TABLE 11
AR-67 Phase II Study Toxicity Compared to Gimatecan Toxicity

| | AR-67 | Gimatecan | |
|---|---|---|---|
| | reGBM | reGBM | reGBM |
| | Single agent | Single agent | Single agent |
| | N = 45 | N = 10 High Dose | N = 10 Low Dose |
| Neutropenia | 13% | 30% | 0% |
| Grades 3-4 | All Grade 3 | | |
| Thrombocytopenia | 4% | 40% | 5% |
| Grades 3-4 | | | |
| Diarrhoea | 0% | 0% | 0% |
| Grades 3-5 | | | |

This comparison of the toxicities seen with AR-67 treatment to Gimatecan treatment shows that AR-67 treatment has reduced toxicities relative to the toxicities induced by Gimatecan treatment. Gimatecan development stopped for hematologic toxicities, and lack of efficacy (PFS6=12%).

Table 12 presents a comparison of the toxicity profiles of AR-67 from this study as a single agent in a reGBM trial, and Lomustine, an alkylating agent also in reGBM trials. See Wick W, et al., 2010, J. Clin. Oncol. 28: 1168-1174; Batchelor TT, rt al., 2013, J. Clin. Oncol. 31: 3212-3218.

TABLE 12
AR-67 Phase II Study Toxicity Compared to Lomustine Toxicity

| | | Lomustine | |
|---|---|---|---|
| | AR-67 | reGBM | reGBM |
| | reGBM | Single agent vs | Single agent/ |
| | Single agent | Enzastaurin N = 92; | placebo |
| | N = 45 | Treated = 84 | N = 64 |
| Neutropenia | 13% | 20% | 3% |
| Grades 3-4 | All grade 3 | | |
| Thrombocytopenia | 4% | 25% | 22% |
| Grades 3-4 | | | |
| Diarrhoea | 0% | NR | 2% |
| Grades 3-5 | | | |
| Nausea | 2% | 0% | NR |
| Grades 3-5 | | | |
| Vomiting | 0% | NR | NR |
| Grades 3-5 | | | |

This comparison of the toxicities seen with AR-67 treatment to Lomustine treatment shows that AR-67 treatment has reduced toxicities relative to the toxicities induced by Lomustine treatment.

While the above disclosure has been described with reference to exemplary embodiments, those of ordinary skill in the art will understand that various changes in form and details may be made without departing from the spirit and scope of the present invention as defined by the following claims.

1.-19. (canceled)

20. A method of treating cancer in a patient, comprising administering to a patient a composition comprising a therapeutically effective amount of AR-67, wherein the method is at least as effective as the administration of a dose of other camptothecins delivered alone or as a combination therapy, and wherein the method reduces the incidence of one or more adverse events.

21. The method of claim 20, wherein the method reduces the incidence of one or more adverse events relative to other camptothecins or to the combination therapy.

22. The method of claim 20, wherein the composition consists essentially of AR-67.

23. The method of claim 20, wherein the method is not a combination therapy with another therapeutically active agent.

24. The method of claim 20, wherein the cancer is selected from the group consisting of glioblastoma multiform, myelodysplastic syndrome, metastatic adenocarcinoma, metastatic carcinoma of the colon or rectum, small cell lung cancer, non-small cell lung cancer, soft tissue sarcoma, squamous cell cancer of head and neck, non-Hodgkin's lymphoma, colorectal cancer, bladder cancer, prostate cancer, duodenal cancer, esophageal cancer, pancreatic cancer, multiple myeloma, and gynecologic tumors.

25. The method of claim 20, wherein the adverse events are selected from the group consisting of neutropenia, thrombocytopenia, diarrhea, fatigue, leucopenia, and anemia.

26. The method of claim 20, wherein the therapeutically effective amount of AR-67 is 1.2-12.4 mg/m2/day.

27. The method of claim 20, wherein the therapeutically effective amount of AR-67 is 1.2-7.5 mg/m2/day.

28. The method of claim 20, wherein the therapeutically effective amount of AR-67 is delivered in the presence of a cremophor.

29. The method of claim 20, wherein the patient is dosed with a therapeutically effective amount of AR-67 on days 1-5 of a 21 day cycle.

30. The method of claim 20, wherein the patient is dosed with a therapeutically effective amount of AR-67 on days 1, 4, 8, 12, and 15, of a 21 day cycle.

31. Composition for use in a method for treating cancer in a patient, the method comprising the method of claim 20.

32. The method of claim 20, wherein the therapeutically effective amount of AR-67 is 2.0-11.0 mg/m2/day, 3.0-9.0 mg/m2/day, 4.0-8.0 mg/m2/day, or 5.0-7.0 mg/m2/day.

33. The method of claim 20, wherein the therapeutically effective amount of AR-67 is 10.0-20.0 mg/m2/day, 15.0-25.0 mg/m2/day, 20.0-30.0 mg/m2/day, or 25.0-35.0 mg/m2/day.

34. The method of claim 20, wherein the therapeutically effective amount of AR-67 is delivered orally or intravenously.

35. The method of claim 20, wherein the therapeutically effective amount of AR-67 is delivered in the absence of a cremophor.

36. The method of claim 20, wherein the therapeutically effective amount of AR-67 is administered as a second-line therapy.

37. The method of claim 20, wherein the patient had failed treatment with a camptothecin other than AR-67 within 90 days of the administering step.

38. The method of claim 27, wherein the camptothecin other than AR-67 is bevacizumab.
